(19) United States
(12) Patent Application Publication
(10) Pub. No.: US 2025/0178996 A1
(43) Pub. Date: Jun. 5, 2025

(54) SGLT2 INHIBITOR INTERMEDIATE V-1 AND USE THEREOF

(71) Applicant: WANBANG BIOPHARMACEUTICALS, Xuzhou, Jiangsu (CN)

(72) Inventors: Keyan WANG, Xuzhou (CN); Shibin WU, Xuzhou (CN); Ruiyan HAN, Xuzhou (CN); Xinmeng ZHU, Xuzhou (CN); Chunlei LI, Xuzhou (CN); Zeng WU, Xuzhou (CN)

(73) Assignee: WANBANG BIOPHARMACEUTICALS, Xuzhou, Jiangsu (CN)

(21) Appl. No.: 18/832,209

(22) PCT Filed: Mar. 15, 2022
(86) PCT No.: PCT/CN2022/080950

§ 371 Date: Jul. 23, 2024

(30) Foreign Application Priority Data
Jan. 26, 2022 (CN) 202210093534.8

(51) Int. Cl. C07C 49/35; C07C 45/59; C07C 45/63; C07C 45/65; C07C 45/86

(52) U.S. Cl. CPC C07C 49/35; C07C 45/59; C07C 45/63; C07C 45/65; C07C 45/86; C07C 2601/14; C07C 2601/16

(57) ABSTRACT

SGLT2 inhibitor intermediate V-1 and the use thereof. The present invention specifically relates to an intermediate as represented by formula V-1 and the use thereof in the preparation of an SCLT2 inhibitor. The intermediate and a method for preparing an SCLT2 inhibitor have the advantages of easily available raw materials, simple operation, short production period, high yield and suitability for industrial production.

### TECHNICAL FIELD

The present invention relates the technical field of medical chemistry, and to an SGLT2 inhibitor intermediate V-1 and use thereof.

### BACKGROUND

Formula (VI) represents a family of aryl, heteroaryl, O-aryl and O-heteroaryl carbasugar compounds, which function as a sodium-dependent glucose transporter 2 (SGLT2) inhibitor. A preparation method therefor is described in the examples of CN104909997B. However, some reaction steps and post-treatments are not suitable for scale-up preparation in industry. For example, column chromatography is used for immobilization and purification in the post-treatment of each step; the reaction temperature in the oxidation step is too low, the reaction rate is slow, and the energy consumption is increased; and the reaction temperature in the fluorination step is high, the fluorinating reagent has the risk of explosion (boiling point 30° C.), and the corrosion of the high-temperature fluorinating reagent steam to the equipment becomes stronger.

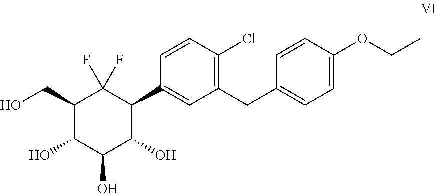 [SMILES: CCOc1ccc(Cc2cc([C@H]3[C@H](O)[C@@H](O)[C@H](O)[C@@H](CO)C3(F)F)ccc2Cl)cc1]

The target molecule is an all carboatomic ring with many chiral centers. The difficulty in synthesizing the compound molecule lies in the construction of the ring of chiral centers. Moreover, for such a polyhydroxy substituted structure, the control chirality is also very risky. Therefore, there is a need to find a simple, low-cost, safe and environmentally friendly preparation method suitable for industrial production with high yield and good quality.

### SUMMARY

The present invention provides a simple, low-cost, safe and environmentally friendly SGLT2 inhibitor intermediate suitable for industrial production with high yield and good quality.

In a first aspect, the present invention provides an SGLT2 inhibitor intermediate V-1, having a chemical name of (2S,3S,4R,5R,6R)-2-(4-chloro-3-(4-ethoxybenzyl)phenyl)-3, 4, 5-trihydroxy-6-(hydroxymethyl)cyclohexan-1-one, and having a structural formula of:

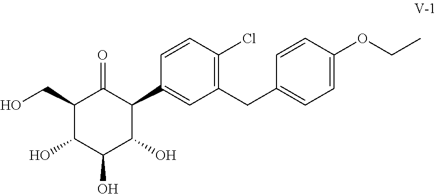 [SMILES: CCOc1ccc(Cc2cc([C@@H]3C(=O)[C@H](CO)[C@@H](O)[C@H](O)[C@H]3O)ccc2Cl)cc1]

In a second aspect, the present invention provides a method for preparing an of an SGLT2 inhibitor intermediate V-1:

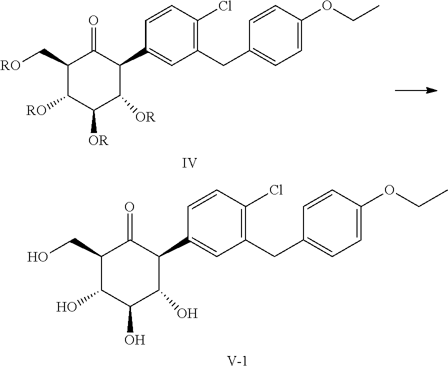

where R is selected from methoxybenzyl, benzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl.

A compound of Formula IV in the present invention can be obtained following the method described in the example in CN104909997B.

In some embodiments, the preparation method of the SGLT2 inhibitor intermediate V-1 of the present invention specifically includes adding the compound of Formula IV and a deprotecting reagent into a solvent, reacting at 10-50° C. for 1-10 hrs, and then carrying out post-treatment, solvent removal, and refining after the reaction is completed to prepare the compound of Formula V-1, that is, (2S,3S,4R,5R,6R)-2-(4-chloro-3-(4-ethoxybenzyl) phenyl)-3, 4, 5-trihydroxy-6-(hydroxymethyl) cyclohexan-1-one.

In some embodiments, the deprotecting reagent is one of palladium on carbon, palladium hydroxide on carbon, tetrabutylammonium bromide, 2,3-dichloro-5,6-dicyanobenzoquinone, hydrogen chloride in methanol, and trifluoroacetic acid, and preferably palladium on carbon.

In some embodiments, the amount of the deprotecting reagent is 5-100 wt % and preferably 10-15 wt % of the amount of the compound of Formula IV.

In some embodiments, the solvent is one of tetrahydrofuran, methanol, dichloromethane, ethanol or acetone or any combination thereof, and preferably tetrahydrofuran.

In some embodiments, the ratio of (2R,3R,4R,5S,6S)-3,4,5-tris(R protecting group-oxy)-2-((R protecting group-oxy)methyl)-6-(4-chloro-3-(4-ethoxybenzyl)phenyl) cyclohexanone to the solvent is 1 g/(5-12) ml.

In some embodiments, the reagent used for refining is methanol, isopropanol, ethanol, acetone, ethyl acetate or acetonitrile, and preferably acetonitrile.

In a third aspect, the present invention provides use of the SGLT2 inhibitor intermediate V-1 in the preparation of an SGLT2 inhibitor of Formula (VI):

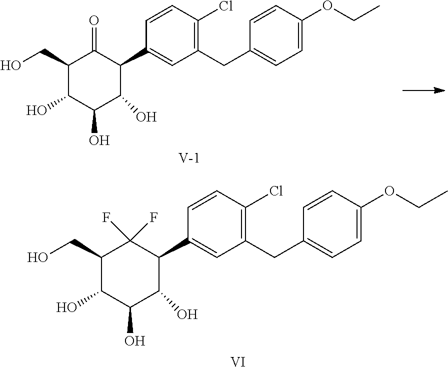

In some embodiments, a method for preparing the compound of Formula VI specifically includes adding the intermediate V-1, a catalyst and triethylamine into a solvent, adding trifluoroacetyl chloride at −5 to 5° C., reacting at room temperature, washing, drying, filtering, then adding diethylaminosulfur trifluoride and anhydrous ethanol, reacting at 15-25° C. for 60-80 hrs, adding a potassium carbonate aqueous solution, and reacting at 20-30° C. for 3-5 hrs, to obtain the compound VI.

In some embodiments, the catalyst in the synthesis of the compound of Formula VI is selected from DMF or DMAP.

In some embodiments, the molar ratio of the intermediate V-1, the catalyst and triethylamine in the synthesis of the compound of Formula VI is 1:(0.1-0.5):(6-8), and preferably 1:(0.1-0.2):(6-8).

In some embodiments, the molar ratio of the intermediate V-1 to trifluoroacetyl chloride in the synthesis of the compound of Formula VI is 1:(5-7).

In some embodiments, the molar ratio of the intermediate V-1 to diethylaminosulfur trifluoride in the synthesis of the compound of Formula VI is 1:(10-20), and preferably 1:15.

In some embodiments, anhydrous ethanol is added in an amount of 1‰-5‰ and preferably 2‰-3‰ by weight of the amount of the intermediate V-1 in the synthesis of the compound of Formula VI.

In a fourth aspect, the present invention further provides a method for preparing an SGLT2 inhibitor of Formula VI:

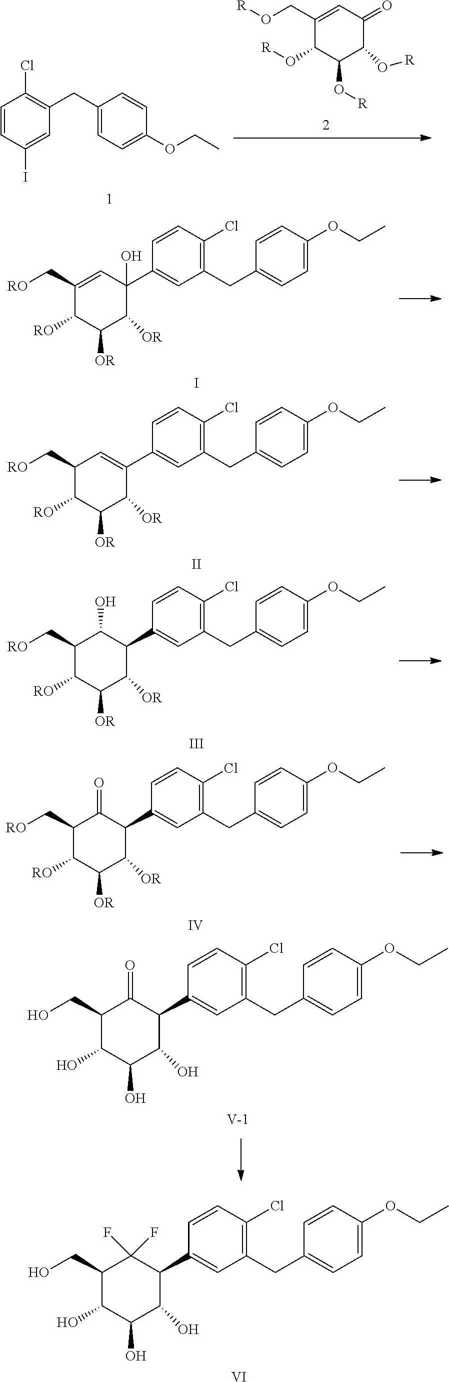

where R is selected from methoxybenzyl, benzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl, and preferably R is selected from benzyl.

In the present invention, the starting material 14-iodo-1-chloro-2-(4-ethoxybenzyl)benzene is commercially available, and the starting material 2 can be obtained following the method described in the example of CN104909997B.

In some embodiments, a method for synthesizing the compound of Formula I includes specifically: adding 4-iodo-1-chloro-2-(4-ethoxybenzyl)benzene (material 1), a material 2, and a metal reagent to a solvent at a molar ratio of (1.05-1.25):1.0:(1.0-1.5), and reacting at −20 to −70° C. for 1-3 hrs.

In some embodiments, the molar ratio of 4-iodo-1-chloro-2-(4-ethoxybenzyl)benzene (material 1), the material 2, and the metal reagent in the synthesis of the compound of Formula I is (1.1-1.2):1:(1.3-1.35).

In some embodiments, the reaction temperature in the synthesis of the compound of Formula I is −25 to −35° C.

In some embodiments, the metal reagent in the synthesis of the compound of Formula I is isopropyl magnesium chloride-lithium chloride or n-butyl lithium.

In some embodiments, the solvent used in the synthesis of the compound of Formula I is one of methyl t-butyl ether, tetrahydrofuran, 2-methyltetrahydrofuran and toluene, or a combination thereof. Preferably, the solvent is tetrahydrofuran or 2-methyltetrahydrofuran. In some embodiments, the ratio of the material 2 to the solvent is 1 g/(5-10) ml.

According to the synthesis method of the compound of Formula I provided in the present invention, the sequence of adding the materials is adjusted, so that the side reaction can be avoided to a great extent. This side reaction leads to an impurity produced by removal of two halogen atoms from the compound 1, which is also the most difficult-to-be-removed impurity with similar polarity in VI.

In some embodiments, a method for synthesizing the compound of Formula II includes specifically: adding the compound of Formula I, triethylsilane, and boron trifluoride etherate to a solvent at a molar ratio of 1.0:(1.5-3.5):(1.1-2.0), reacting at −30 to 0° C. for 1-2 hrs, washing the reaction solution with alkaline water after the reaction is completed, discarding the aqueous layer, removing the solvent, and refining to prepare the compound of Formula II.

In some embodiments, the molar ratio of the compound of Formula I, triethylsilane, and boron trifluoride etherate in the synthesis of the compound of Formula II is 1.0:(2.1-2.7) (1.45-1.8).

In some embodiments, the reaction temperature in the synthesis of the compound of Formula II is −28 to −15° C.

In some embodiments, the solvent used in the synthesis of the compound of Formula II is isopropyl acetate, cyclohexane, dichloromethane, isopropyl ether or acetonitrile, and preferably the solvent is dichloromethane. In some embodiments, the ratio of the compound of Formula I to the solvent is 1 g/(2-15) ml.

In some embodiments, the alkaline water in the synthesis of the compound of Formula II is a sodium bicarbonate solution, a sodium carbonate solution or a sodium hydroxide solution, and preferably a sodium bicarbonate solution.

In some embodiments, the reagent used for refining in the synthesis of the compound of Formula II is one of acetonitrile, isopropanol, methanol, ethanol, isopropyl ether, ethyl acetate or n-heptane, or a combination thereof, and preferably methanol.

According to the method for synthesizing the compound of Formula II provided in the present invention, recrystallization is used in place of column chromatography for purification, which is more conducive to scale-up production in industry.

In some embodiments, a method for synthesizing the compound of Formula III specifically includes: adding the compound of Formula II and a reducing agent to a solvent at a molar ratio of 1.0:(1.1-2.5), and reacting at 40-70° C. for 1-7 hrs; cooling the reaction solution to −5 to 30° C. after the reaction is completed, and further oxidizing for 12-15 hrs at 15-40° C. at a molar ratio of the compound of Formula II:sodium hydroxide:H₂O₂ of 1.0:(3-10):(3-10), followed by washing, extraction, concentration, and refining of the reaction solution, to obtain the compound of Formula III.

In some embodiments, in the synthesis of the compound of Formula III, the molar ratio of the compound of Formula II to the reducing agent is 1.0:(1.4-1.6).

In some embodiments, the reaction temperature in the synthesis of the compound of Formula III is 60-70° C. In some embodiments, after the reaction is completed, the reaction solution is cooled to 0-15° C.

In some embodiments, the molar ratio of the compound of Formula II:sodium hydroxide:H₂O₂ in the synthesis of the compound of Formula III is 1.0:(7.5-9.5):(8.0-9.0).

In some embodiments, the reaction temperature for further oxidation in the synthesis of the compound of Formula III is 25 to 33° C.

In some embodiments, the reducing agent in the synthesis of the compound of Formula III is borane dimethyl sulfide, sodium borohydride, pinacol borane, 8-methoxy-9-borabicyclic[3.3.1]nonane or borane tetrahydrofuran complex, and preferably borane dimethyl sulfide or borane tetrahydrofuran complex.

In some embodiments, the solvent used in the synthesis of the compound of Formula III is one of toluene, tetrahydrofuran, 1,4-dioxane and dichloromethane, or a combination thereof, and preferably tetrahydrofuran; In some embodiments, the ratio of the compound of Formula II to the solvent is 1 g/(6-14) ml.

In some embodiments, the reagent used for refining in the synthesis of the compound of Formula III is one of acetonitrile, n-heptane, methyl tert-butyl ether, ethanol, isopropyl ether and ethyl acetate or a combination thereof, and preferably isopropyl ether.

According to the method for synthesizing the compound of Formula III provided in the present invention, recrystallization is used in place of column chromatography for purification, which is more conducive to scale-up production in industry.

In some embodiments, a method for synthesizing the compound of Formula IV includes specifically: adding the compound of Formula III and an oxidant to a solvent at a molar ratio of 1.0:(1.0-4.0), and reacting at −5 to 30° C. for 1-5 hrs; and washing the reaction solution with water containing a reducing agent after the reaction is completed, discarding the aqueous layer, removing the solvent, and refining to produce the compound of Formula IV.

In some embodiments, in the synthesis of the compound of Formula IV, the molar ratio of the compound of Formula III to the oxidant is 1.0:(1.5-1.8).

In some embodiments, the reaction temperature in the synthesis of the compound of Formula IV is 5 to 15° C.

In some embodiments, the oxidant used in the synthesis of the compound of Formula IV is manganese dioxide, Dess-Martin oxidant, Jones reagent or 2,2,6,6-tetramethylpiperidine oxide, and preferably Dess-Martin oxidant.

In some embodiments, the solvent used in the synthesis of the compound of Formula IV is one of isopropyl ether, dichloromethane, tetrahydrofuran, toluene, and acetonitrile, or a combination thereof, and preferably dichloromethane. In some embodiments, the ratio of the compound of Formula III to the solvent is 1 g/(3-15) ml.

In some embodiments, the reagent used for refining in the synthesis of the compound of Formula IV is isopropyl ether, isopropyl alcohol, ethanol, acetone, ethyl acetate or tetrahydrofuran, and preferably isopropyl alcohol.

According to the method for synthesizing the compound of Formula IV provided in the present invention, the reaction time is about 1-5 hrs, which is significantly reduced compared with the reaction time of 12-18 hrs in the previous research.

In some embodiments, a method for synthesizing the compound of Formula V-1 includes specifically: adding the compound of Formula IV and a deprotecting reagent to a solvent, reacting at 10-50° C. for 1-10 hrs, and then carrying out post-treatment, solvent removal, and refining after the reaction is completed to obtain the compound of Formula V-1.

In some embodiments, the deprotecting reagent in the synthesis of the compound of Formula V-1 is one of palladium on carbon, palladium hydroxide on carbon, tetrabutylammonium bromide, 2,3-dichloro-5,6-dicyanobenzoquinone, hydrogen chloride in methanol, and trifluoroacetic acid, and preferably palladium on carbon.

In some embodiments, the amount of the deprotecting reagent used in the synthesis of the compound of Formula V-1 is 5-100 wt % and preferably 10-18 wt % of the amount of the compound of Formula IV.

In some embodiments, the solvent in the synthesis of the compound of Formula V-1 is one of tetrahydrofuran, methanol, dichloromethane, ethanol or acetone or any combination thereof, and preferably tetrahydrofuran.

In some embodiments, in the synthesis of the compound of Formula V-1, the ratio of the compound of Formula IV to the solvent is 1 g/(5-12) ml.

In some embodiments, the reagent used for refining in the synthesis of the compound of Formula V-1 is methanol, isopropyl alcohol, ethanol, acetone, ethyl acetate or acetonitrile, and preferably acetonitrile.

In some embodiments, a method for synthesizing the compound of Formula VI specifically includes adding the intermediate V-1, a catalyst and triethylamine into a solvent, adding trifluoroacetyl chloride at −5 to 5° C., reacting at room temperature, washing, drying, filtering, then adding diethylaminosulfur trifluoride and anhydrous ethanol, reacting at 15-25° C. for 60-80 hrs, adding a potassium carbonate aqueous solution, and reacting at 20-30° C. for 3-5 hrs, to obtain the compound VI.

In some embodiments, the catalyst in the synthesis of the compound of Formula VI is selected from DMF or DMAP.

In some embodiments, the molar ratio of the intermediate V-1, the catalyst and triethylamine in the synthesis of the compound of Formula VI is 1:(0.1-0.5):(6-8), and preferably 1:(0.1-0.2):(6-8).

In some embodiments, the molar ratio of the intermediate V-1 to trifluoroacetyl chloride in the synthesis of the compound of Formula VI is 1:(5-7).

In some embodiments, the molar ratio of the intermediate V-1 to diethylaminosulfur trifluoride in the synthesis of the compound of Formula VI is 1:(10-20), and preferably 1:15.

In some embodiments, anhydrous ethanol is added in an amount of 1‰-5‰ and preferably 2‰-3‰ by weight of the amount of the intermediate V-1 in the synthesis of the compound of Formula VI.

### Beneficial Effects

The present invention provides an intermediate compound V-1 having a structure that is an all carboatomic ring with multiple chiral centers and use thereof in the preparation of VI. The preparation process of VI provided in the present invention is simple, low in cost, safe and environment-friendly, and suitable for use in industrial production. The purity of the final product prepared with the intermediate compound V-1 is over 97%, the yield can reach about 80%, and the chirality is well controlled, which are conducive to providing a high-quality SGLT2 inhibitor compound at a low cost.

### BRIEF DESCRIPTION OF THE DRAWINGS

FIG. 1 is a ¹H NMR spectrum of a compound V-1;

FIG. 2 is a ¹³C NMR spectrum of the compound V-1;

FIG. 3 is an IR spectrum of the compound V-1;

FIG. 4 is an UV spectrum of the compound V-1;

FIG. 5 is a LC-MS spectrum of the compound V-1; and

FIG. 6 is an HPLC chromatogram of the compound V-1.

### DETAILED DESCRIPTION

The following examples are provided for a better understanding of the present invention; however, the present invention is not limited thereto. The methods given in examples below are all conventional methods, unless it is otherwise stated. Test materials used in the following examples are commercially available or can be prepared through a method reported in the literatures, unless otherwise specified.

### Example 1. Preparation of Compound V-1 where R is Benzyl Group

### 1) (2R,3S,4R)-2,3,4-tris(benzyloxy)-5-((benzyloxy)methyl)-4′-chloro-3′-(4-ethoxybenzyl)-1,2,3,4-tetrahydro-[1,1′-diphenyl]-1-ol (Formula I)

Anhydrous tetrahydrofuran (98 g, 110 ml) and then 4-iodo-1-chloro-2-(4-ethoxybenzyl)benzene (15.6 g, 41.86 mmol, 1.12 eq) were added to a clean and dry reaction flask, stirred until the solid was dissolved, purged with nitrogen, and cooled to −25 to −30° C. A solution of isopropyl magnesium chloride/lithium chloride (35.34 g, 48.39 mmol, 1.3 eq) was added dropwise. After that, the reaction was continued at −25 to −30° C. further for 1 h. The remaining of the starting raw material was detected by HPLC. After the reaction was completed, (4R,5S,6R)-4,5,6-tris(benzyloxy)-3-((benzyloxy)methyl) cyclohex-2-en-1-one (19.9 g, 37.22 mmol, 1.0 eq) was dissolved in tetrahydrofuran (20 g), and added dropwise to the reaction flask at a temperature controlled to −25 to −30° C. After that, the reaction solution was stirred for 1 hr while the temperature was maintained, and the reaction progress was detected by HPLC. After the reaction was completed, the reaction solution was quenched by slowly adding to an ammonium chloride solution (130 g, 10%), and extracted by adding ethyl acetate (110 g). The organic phase was separated, washed with saturated sodium chloride (150 g), dried, and concentrated at 40-45° C. under reduced pressure until no fraction was flowed out. A brown oil (31.1 g, yield 106.9%) was obtained.

### 2) (2R,3S,4R,5R)-2,3,4-tris(benzyloxy)-5-((benzyloxy)methyl)-4′-chloro-3′-(4-ethoxybenzyl)-2,3,4,5-tetrahydro-1,1′-biphenyl (Formula II)

Dichloromethane (170 g, 130 ml) and then (2R,3S,4R)-2,3,4-tris(benzyloxy)-5-((benzyloxy)methyl)-4′-chloro-3′-(4-ethoxybenzyl)-1,2,3,4-tetrahydro-[1,1′-diphenyl]-1-ol (29 g, 37.11 mmol, 1.0 eq) were added to a clean and dry reaction flask, purged with nitrogen, and then cooled to −25 to −15° C. Triethylsilane (9.7 g, 83.42 mmol, 2.25 eq) was added dropwise. After that, boron trifluoride etherate (7.9 g, 55.67 mmol, 1.50 eq) was further added dropwise. Then, the reaction was continued for 1 hr while the temperature was held. The reaction process was detected by HPLC. After the reaction was completed, the temperature was controlled to 15° C. or below. Purified water (58 g) was added to the reaction solution. The organic phase was washed to neutral with a saturated sodium bicarbonate aqueous solution (88 g), and then with saturated brine (88 g). The organic phase was collected. The organic phase was then concentrated at 37±3° C. under reduced pressure until no fraction was flowed out. The concentrated oil was added with methanol (110 g), heated to 70-80° C., then cooled to 10-15° C., and crystallized by stirring for 16 hrs. The product was filtered under suction, rinsed with methanol (30 g), and then further filtered under suction to obtain a wet product (20.5 g). The wet product was dried in the air at 50±3° C. for 12 hrs to obtain a pale yellow solid (17.04 g, purity 95.4%, yield 60.0%).

### 3) (1R,2S,3R,4R,5S,6R)-3,4,5-tris(benzyloxy)-2-((benzyloxy)methyl)-6-(4-chloro-3-(4-ethoxybenzyl)phenyl)cyclohexanol (Formula III)

Anhydrous tetrahydrofuran (130.0 g, 146 ml) and then (2R,3S,4R,5R)-2,3,4-tris(benzyloxy)-5-((benzyloxy) methyl)-4′-chloro-3′-(4-ethoxybenzyl)-2,3,4,5-tetrahydro-1,1′-biphenyl (15.7 g, 20.51 mmol, 1.00 eq) were added to a clean and dry reaction flask, and purged with nitrogen. After the system was completely dissolved, a solution of borane dimethyl sulfide (2.4 g, 30.77 mmol, 1.50 eq) was slowly added while the temperature was controlled to 20-30° C. After that, the reaction solution was slowly heated to 60-70° C. and reacted for 2.5 hrs. The reaction progress was detected by HPLC. After the reaction was completed, the reaction solution was cooled to 0-10° C., and a 5 mol/L solution of sodium hydroxide (39.6 g, 167.0 mmol, 8.15 eq) and then a 30% solution of hydrogen peroxide (18.7 g 165.0 mmol, 8.04 eq) were slowly added dropwise while the temperature was maintained. After that, the reaction solution was slowly heated to 25-30° C. and reacted further for 15 hrs. A saturated ammonium chloride solution (150 g) and a saturated sodium chloride solution (78 g) were added to the reaction solution and stirred for 1 hr. The solution was allowed to stand still, and the organic phase was separated and collected. The aqueous phase was extracted once with ethyl acetate (75 g). The organic phases were combined. The organic phase was washed with a 10% Na₂SO₃ solution (80 g). The organic phase was collected, and had no color change when tested with potassium iodide test paper. The organic phase was concentrated at 40-45° C. under reduced pressure, until no fraction was flowed out. The resulting oil was added with isopropyl ether (35 g), stirred at 20-25° C. for 12 hrs, and filtered under suction. The wet product was collected and dried under vacuum at 45±3° C. for 10 hrs, to obtain an off-white solid (8.5 g, purity 92.1%, yield 52.9%).

### 4) Preparation of (2R,3R,4R,5S,6S)-3,4,5-tri(benzyloxy)-2-((benzyloxy)methyl)-6-(4-chloro-3-(4-ethoxybenzyl)phenyl)cyclohexanone of Formula IV

Dichloromethane (75 g, 58 ml) and (1R,2S,3R,4R, 5S,6R)-3,4,5-tris(benzyloxy)-2-((benzyloxy)methyl)-6-(4-chloro-3-(4-ethoxybenzyl)phenyl)cyclohexanol (7 g, 8.94 mmol, 1.0 eq) were added to a dry and clean reaction flask, and stirred until the solid was dissolved. The reaction solution was cooled to 5-12° C., added with Dess-Martin oxidant (5.8 g, 13.67 mmol, 1.53 eq), and reacted for 2.5 hrs while the temperature was maintained at 8-15° C. The reaction progress was detected by HPLC. After the reaction was completed, the reaction solution was slowly added to an aqueous solution (65 mL) containing anhydrous Na₂SO₃ (6 g) and NaHCO₃ (5 g), and stirred for 30 min. The solution was allowed to stand still, and the organic phase was separated and collected. The aqueous phase was extracted with dichloromethane (30 g). The organic phases were combined. The organic phase was washed with purified water (30 g). The organic phase was separated and, collected, dried over anhydrous sodium sulfate (7 g) for 30 min, and filtered. The filtrate was collected, and concentrated under reduced pressure at 30±3° C., to obtain a yellowish-brown oil. Isopropyl alcohol (10 g) was added to the oil, heated to 70-80° C. to dissolve it, then slowly cooled, and crystallized by stirring for 16 hrs while the temperature was maintained at 20-25° C. The reaction solution was filtered. The material was collected and dried in a vacuum oven at 50±3° C. for 10 hrs, to obtain (2R,3R,4R,5S,6S)-3,4,5-tri(benzyloxy)-2-((benzyloxy)methyl)-6-(4-chloro-3-(4-ethoxybenzyl)phenyl)cyclohexanone (5.5 g, purity 94.7%, yield 78.7%).

### 5) Preparation of (2S,3S,4R,5R,6R)-2-(4-chloro-3-(4-ethoxybenzyl) phenyl)-3, 4, 5-trihydroxy-6-(hydroxymethyl) cyclohexan-1-one of Formula V-1

(2R,3R,4R,5S,6S)-3,4,5-tri(benzyloxy)-2-((benzyloxy)methyl)-6-(4-chloro-3-(4-ethoxybenzyl)phenyl)cyclohexanone (5 g, 6.40 mmol, 1.0 eq), and tetrahydrofuran (25 g, 28 ml) were added to a 100 mL hydrogenation reactor, and stirred until the solid was completely dissolved. Then, 5% palladium on carbon (0.7 g) was added and stirred until uniform. Stirring was started at a speed of 25 Hz. The reactor was purged three times with nitrogen and then three times with hydrogen. The temperature was controlled at 20-30° C., the hydrogen pressure was 0.09-0.13 MPa, and the reaction time was 70 min. The reaction progress was detected by HPLC. After the reaction was completed, hydrogen was vented, and the reactor was purged three times with nitrogen. Palladium on carbon was removed by filtration, and the filter cake was washed with tetrahydrofuran (4 g). The filtrates were combined, concentrated to dryness under reduced pressure at 45±3° C., and acetonitrile (8.5 g) were added and heated to reflux. After complete dissolution, the solution was slowly cooled, and crystallized for 2 hrs while the temperature was maintained at 40-50° C. The crystallization was continued by cooling to 0-10° C., with stirring at 0-10° C. for 1 hr. After filtration, the filter cake was dried under vacuum at 50±3° C. for 8 hrs, to obtain a white solid as a solid (2.2 g, purity 98.4%, yield 81.7%).

### 6) Preparation of (1R,2R,3S,4S,6R)-4-(4-chloro-3-(ethoxybenzyl) phenyl)-5,5-difluoro-6-(hydroxymethyl)cyclohexan-1,2,3-triol of Formula VI

(2S,3S,4R,5R,6R)-2-(4-chloro-3-(4-ethoxybenzyl) phenyl)-3, 4, 5-trihydroxy-6-(hydroxymethyl) cyclohexan-1-one (5 g, 11.9 mmol, 1.0 eq), dichloromethane (48 g, 15 ml), DMF (0.09 g, 1.2 mmol, 0.1 eq) and triethyl amine (9 g, 89.3 mmol, 7.5 eq) were added to a clean and dry reaction flask, and purged and protected with nitrogen. After cooling to −5° C. to 5° C., trifluoroacetyl chloride (11 g, 83.3 mmol, 7.0 eq) was slowly added dropwise. After that, the reaction was continued at room temperature for 8 hrs. The reaction endpoint was monitored by HPLC. After the reaction was completed, the reaction solution was diluted with dichloromethane (48 g, 15 ml), and washed once with a 2 N hydrochloric acid aqueous solution, once with a saturated sodium bicarbonate aqueous solution, and once with saturated brine. The organic phase was dried overnight. After filtration, the filtrate was transferred to a dry reaction flask, then diethylaminosulfur trifluoride (27 g, 168 mmol, 15.0 eq) and anhydrous ethanol (0.015 g, 30‰ M_{SM}) were added, heated to 20° C. and reacted for 72 hrs. The reaction solution was quenched with iced water, neutralized, and washed. The organic phase was separated, and concentrated to half of the volume. A 5% potassium carbonate aqueous solution (15 ml) was added and stirred at 20-25° C. for 4 hrs. After filtration, the filter cake was slurried in water and filtered again to obtain a crude product of Formula (VI). The crude product was added to acetonitrile (15 ml), and heated to reflux. After complete dissolution, the solution was slowly cooled, and crystallized for 1 hr while the temperature was maintained at 40-50° C. The reaction solution was cooled to 0-10° C., and continuously stirred at 0-10° C. for 1 hr. After filtration, the filter cake was dried under vacuum at 50±3° C. for 8 hrs, to obtain a white solid as a solid (4.48 g, purity 98.8%, yield 85.0%).

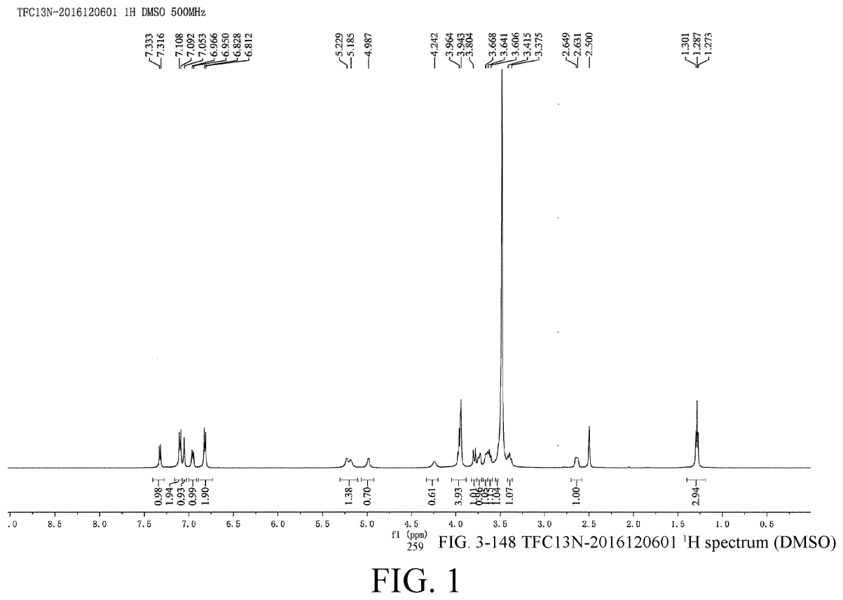

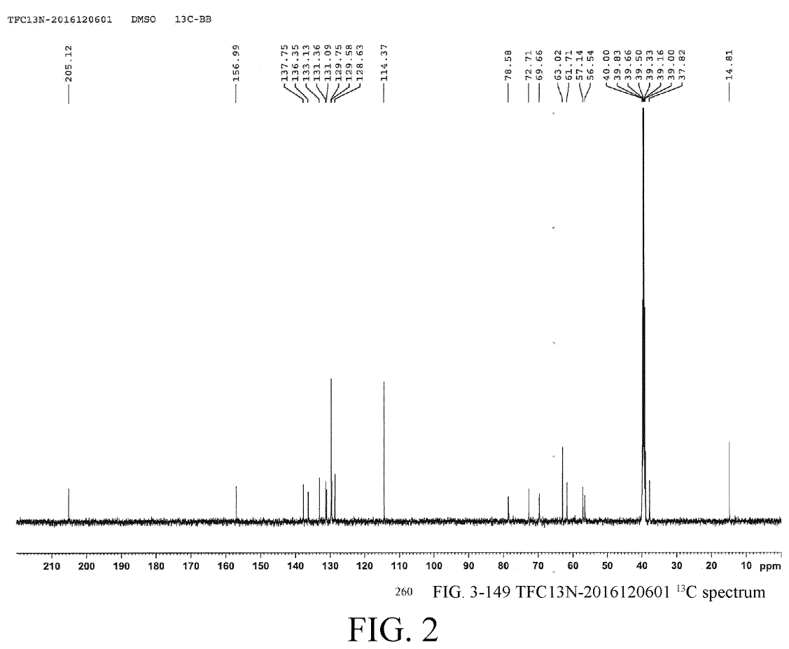

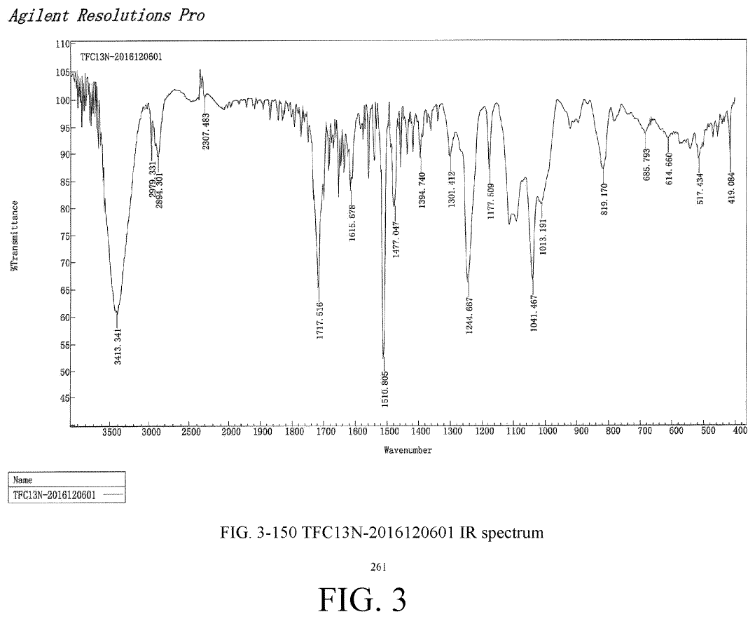

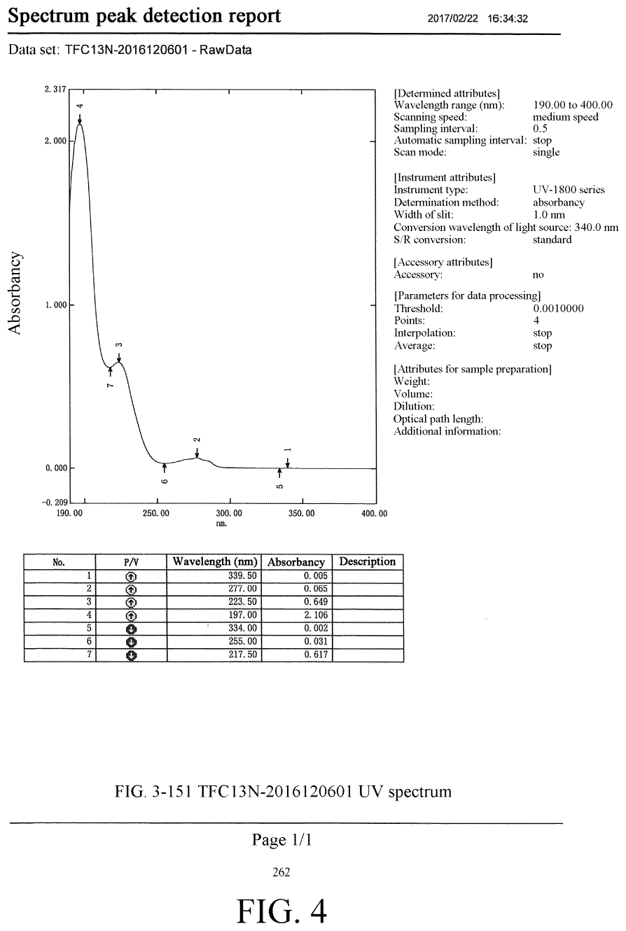

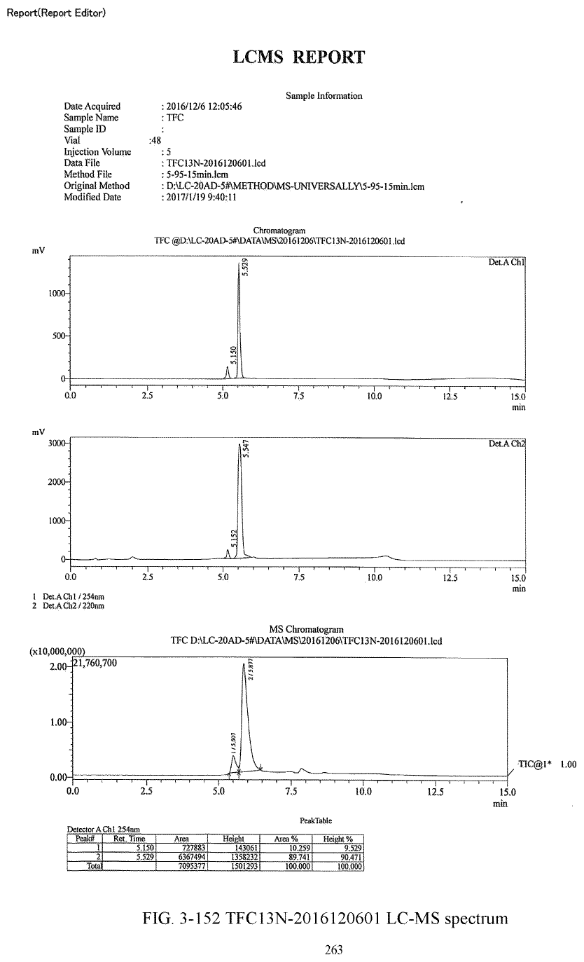

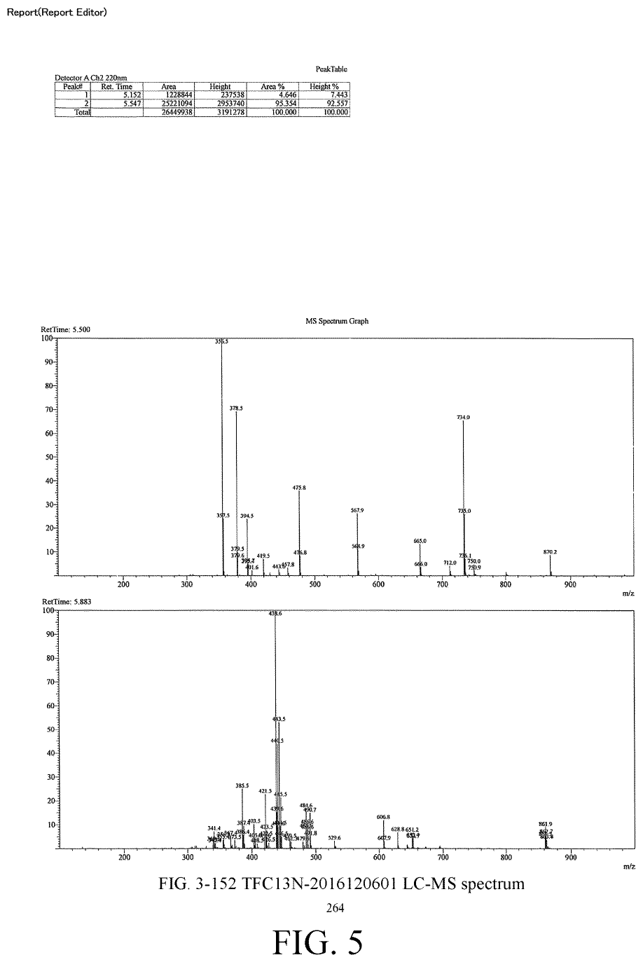

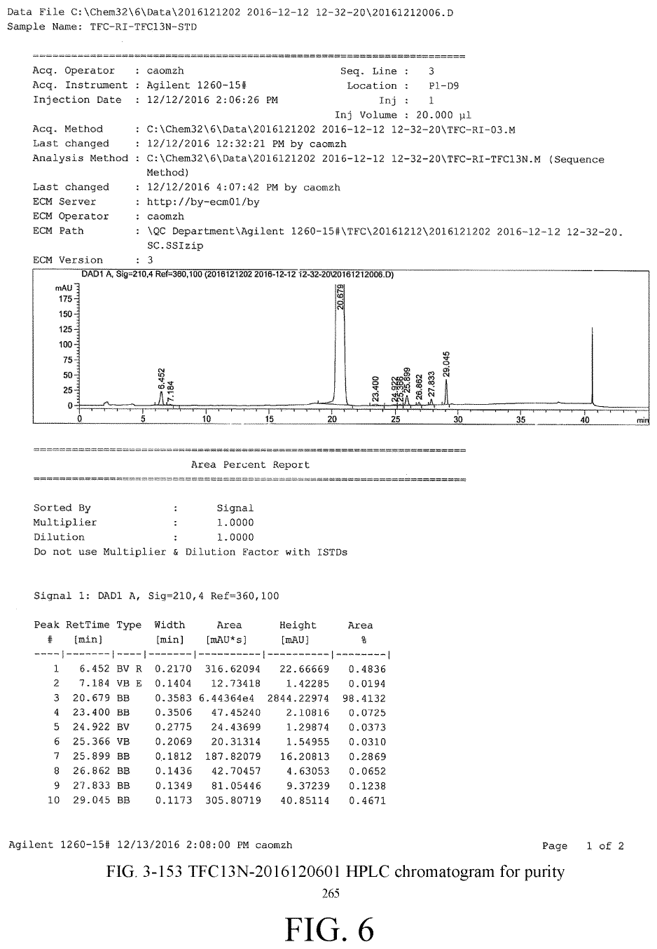

The characterization data of the compound V-1 is shown in FIGS. 1-6 and Table 1.

TABLE 1
Characterization data of the compound V-1

| Item | Feature data | Feature attribution |
|---|---|---|
| ¹H NMR | 7.316-7.333 (d, 1H, J = 8.5 Hz), 7.092-7.108 (d, | Phenyl ring proton |
| (D6-DMSO, δ, | 2H, J = 8 Hz), 7.053 (s, 1H), 6.950-6.966 (d, 1H, | |
| ppm), 400 MHz | J = 8 Hz), 6.812-6.828 (d, 2H, J = 8 Hz) | |
| | 5.229 (br, 1H), 5.185 (br, 1H), 4.987 (br, 1H), | Hydroxyl proton |
| | 4.242 (br, 1H) | |
| | 3.943-3.977 (m, 4H), 3.781-3.804 (d, 1H, | |
| | J = 11.5), 3.727-3.745 (d, 1H, J = 9 Hz), 3.668 (m, | |
| | 2H), 3.524 (m, 1H), 3.415 (m, 1H), 2.649 (m, 1H) | |
| | 1.287(t, 3H, J = 7 Hz) | Methyl proton |
| ¹³C NMR | 205.12 | Ketone carbonyl carbon |
| (D6-DMSO, δ, | 156.99, 137.75, 136.35, 133.13, 131.36, | Phenyl ring carbon |
| ppm), 100 MHz | 131.09, 129.75, 129.58, 128.63, 114.37 | |
| | 78.58, 72.71, 61.71, 57.14, 56.54 | Carbon in partially |
| | | saturated region |
| | 63.02, 14.81 | Ethyl carbon of ethoxy |
| | 37.82 | Benzyl carbon of |
| | | ethoxybenzyl |
| LCMS | m/z = 443 | M + Na⁺ |
| IR | 3413.341 | OH stretching vibration |
| | 2979.331, 2894.301 | Methyl/methylene |
| | | stretching vibration |
| | 1717.516 | Carbonyl C═O |
| | | stretching vibration |
| | 1615.678, 1510.805 | Benzene ring skeleton |
| | | vibration |
| | 1477.047, 1394.740 | Methyl/methylene |
| | | bending vibration |
| | 1244.667, 1177.509 | C—O stretching |
| | | vibration in alcohol or |
| | | ether |
| | 819.170 | C—H bending vibration |
| | | of benzene ring |
| UV | 277.00 | B band |
| | 223.50 | K band |
| | 197.00 | E band |

### Example 2. Preparation of Compound V-1 where R is p-Methoxybenzyl

### 1) (2R,3S,4R)-2,3,4-tri(p-methoxybenzyloxy)-5-((p-methoxybenzyloxy)methyl)-4′-chloro-3′-(4-ethoxybenzyl)-1,2,3,4-tetrahydro-[1,1′-diphenyl]-1-ol (Formula I)

Anhydrous tetrahydrofuran (150 g, 170 ml) and then 4-iodo-1-chloro-2-(4-ethoxybenzyl)benzene (22.0 g, 59.04 mmol, 1.20 eq) were added to a clean and dry reaction flask, stirred until the solid was dissolved, purged with nitrogen, and cooled to −25 to −30° C. A solution of isopropyl magnesium chloride/lithium chloride (46.4 g, 63.53 mmol, 1.30 eq) was added dropwise. After that, the reaction was continued at −25 to −30° C. further for 1 h. The remaining of the starting raw material was detected by HPLC. After the reaction was completed, (4R,5S,6R)-4,5,6-tris(p-methoxybenzyloxy)-3-((p-methoxybenzyloxy) methyl)cyclohex-2-en-1-one (32.0 g, 48.87 mmol, 1.0 eq) was dissolved in tetrahydrofuran (35 g), and added dropwise to the reaction flask at a temperature controlled to −25 to −35° C. After that, the reaction solution was stirred for 1.5 hrs while the temperature was maintained, and the reaction progress was detected by HPLC. After the reaction was completed, the reaction solution was quenched by slowly adding to an ammonium chloride solution (200 g, 10%), and extracted by adding ethyl acetate (150 g). The organic phase was separated, washed with saturated sodium chloride (230 g), dried, and concentrated at 40-45° C. under reduced pressure until no fraction was flowed out. A brown oil (45.1 g, yield 102.4%) was obtained.

### 2) (2R,3S,4R,5R)-2,3,4-tri(p-methoxybenzyloxy)-5-((p-methoxybenzyloxy) methyl)-4′-chloro-3′-(4-ethoxybenzyl)-2,3,4,5-tetrahydro-1,1′-biphenyl (Formula II)

Dichloromethane (200 g, 154 ml) and then (2R,3S,4R)-2,3,4-tri(p-methoxybenzyloxy)-5-((p-methoxybenzyloxy)methyl)-4′-chloro-3′-(4-ethoxybenzyl)-1,2,3,4-tetrahydro-[1,1′-diphenyl]-1-ol (35 g, 38.83 mmol, 1.0 eq) were added to a clean and dry reaction flask, purged with nitrogen, and then cooled to −28 to −15° C. Triethylsilane (12.1 g, 104.06 mmol, 2.68 eq) was added dropwise. After that, boron trifluoride etherate (9.6 g, 67.63 mmol, 1.74 eq) was further added dropwise. Then, the reaction was continued for 2 hrs while the temperature was held. The reaction process was detected by HPLC. After the reaction was completed, the temperature was controlled to 10° C. or below. Purified water (70 g) was added to the reaction solution. The organic phase was washed to neutral with a saturated sodium bicarbonate aqueous solution (110 g), and then with saturated brine (110 g). The organic phase was collected. The organic phase was then concentrated at 37±3° C. under reduced pressure until no fraction was flowed out. The concentrated oil was added with methanol (140 g), heated to 70-80° C., then cooled to 10-18° C., and crystallized by stirring for 16 hrs. The product was filtered under suction, rinsed with methanol (40 g), and then further filtered under suction to obtain a wet product (25.1 g). The wet product was dried in the air at 50±3° C. for 10 hrs to obtain a pale yellow solid (18.9 g, purity 94.8%, yield 55.0%).

### 3) (1R,2S,3R,4R,5S,6R)-3,4,5-tri(p-methoxybenzyloxy)-2-((p-methoxybenzyloxy) methyl)-6-(4-chloro-3-(4-ethoxybenzyl)phenyl)cyclohexanol (Formula III)

Anhydrous tetrahydrofuran (165.0 g, 185 ml) and then (2R,3S,4R,5R)-2,3,4-tri(p-methoxybenzyloxy)-5-((p-methoxybenzyloxy) methyl)-4′-chloro-3′-(4-ethoxybenzyl)-2,3,4,5-tetrahydro-1,1′-biphenyl (18.9 g, 21.34 mmol, 1.0 eq) were added to a clean and dry reaction flask, and purged with nitrogen. After the system was completely dissolved, a solution of borane dimethyl sulfide (2.6 g, 32.50 mmol, 1.52 eq) was slowly added while the temperature was controlled to 20-28° C. After that, the reaction solution was slowly added to 60-68° C. and reacted for 2 hrs. The reaction progress was detected by HPLC. After the reaction was completed, the reaction solution was cooled to 0-8° C., and a 5 mol/L solution of sodium hydroxide (40.2 g, 169.6 mmol, 7.95 eq) and then a 30% solution of hydrogen peroxide (20.4 g 180.0 mmol, 8.43 eq) were slowly added dropwise while the temperature was maintained. After that, the reaction solution was slowly heated to 25-33° C. and reacted further for 12 hrs. A saturated ammonium chloride solution (185 g) and a saturated sodium chloride solution (95 g) were added to the reaction solution and stirred for 1 hr. The solution was allowed to stand still, and the organic phase was separated and collected. The aqueous phase was extracted once with ethyl acetate (90 g). The organic phases were combined. The organic phase was washed with a 10% Na₂SO₃ solution (100 g). The organic phase was collected, and had no color change when tested with potassium iodide test paper. The organic phase was concentrated at 40-44° C. under reduced pressure, until no fraction was flowed out. The resulting oil was added with isopropyl ether (41.5 g), stirred at 20-25° C. for 10 hrs, and filtered under suction. The wet product was collected and dried under vacuum at 45±3° C. for 12 hrs, to obtain an off-white solid (10.3 g, purity 93.4%, yield 53.4%).

### 4) Preparation of (2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone of Formula IV

Dichloromethane (90 g, 70 ml) and (1R,2S,3R,4R,5S,6R)-3,4,5-tri (p-methoxybenzyloxy)-2-((p-methoxybenzyloxy)methyl)-6-(4-chloro-3-(4-ethoxybenzyl)phenyl)cyclohexanol (8.3 g, 9.19 mmol, 1.0 eq) were added to a dry and clean reaction flask, and stirred until the solid was dissolved. The reaction solution was cooled to 5-15° C., added with Dess-Martin oxidant (6.5 g, 15.33 mmol, 1.66 eq), and reacted for 3 hrs while the temperature was maintained at 8-12° C. The reaction progress was detected by HPLC. After the reaction was completed, the reaction solution was slowly added to an aqueous solution (80 mL) containing anhydrous Na₂SO₃ (7.3 g) and NaHCO₃ (6 g), and stirred for 30 min. The solution was allowed to stand still, and the organic phase was separated and collected. The aqueous phase was extracted with dichloromethane (38 g). The organic phases were combined. The organic phase was washed with purified water (40 g). The organic phase was separated and, collected, dried over anhydrous sodium sulfate (8.2 g) for 30 min, and filtered. The filtrate was collected, and concentrated under reduced pressure at 30±4° C., to obtain a yellowish-brown oil. Isopropyl alcohol (15 g) was added to the oil, heated to 70-78° C. to dissolve it, then slowly cooled, and crystallized by stirring for 12 hrs while the temperature was maintained at 20-28° C. The reaction solution was filtered. The material was collected and dried in a vacuum oven at 50±3° C. for 12 hrs, to obtain (2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone (6.8 g, purity 95.1%, yield 82.1%).

### 5) Preparation of (2S,3S,4R,5R,6R)-2-(4-chloro-3-(4-ethoxybenzyl) phenyl)-3, 4, 5-trihydroxy-6-(hydroxymethyl) cyclohexan-1-one of Formula V-1

(2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone (5 g, 5.55 mmol, 1.0 eq) and tetrahydrofuran (40 g, 45 ml) were added to a 100 mL hydrogenation reactor, and stirred until the solid was completely dissolved. Then, 5% palladium on carbon (0.85 g) was added and stirred until uniform. Stirring was started at a speed of 28 Hz. The reactor was purged three times with nitrogen and then three times with hydrogen. The temperature was controlled at 20-28° C., the hydrogen pressure was 0.09-0.15 MPa, and the reaction time was 80 min. The reaction progress was detected by HPLC. After the reaction was completed, hydrogen was vented, and the reactor was purged three times with nitrogen. Palladium on carbon was removed by filtration, and the filter cake was washed with tetrahydrofuran (6 g). The filtrates were combined, concentrated to dryness under reduced pressure at 45±2° C., and acetonitrile (11 g) were added and heated to reflux. After complete dissolution, the solution was slowly cooled, and crystallized for 2.5 hrs while the temperature was maintained at 40-48° C. The crystallization was continued by cooling to 0-8° C., with stirring at 0-10° C. for 1.5 hrs. After filtration, the filter cake was dried under vacuum at 50±2° C. for 10 hrs, to obtain a white solid as a solid (1.9 g, purity 99.1%, yield 81.3%).

### 6) Preparation of (1R,2R,3S,4S,6R)-4-(4-chloro-3-(ethoxybenzyl) phenyl)-5,5-difluoro-6-(hydroxymethyl)cyclohexan-1,2,3-triol of Formula VI

(2S,3S,4R,5R,6R)-2-(4-chloro-3-(4-ethoxybenzyl) phenyl)-3, 4, 5-trihydroxy-6-(hydroxymethyl) cyclohexan-1-one (5 g, 11.9 mmol, 1.0 eq), dichloromethane (48 g, 15 ml), DMAP (0.15 g, 1.2 mmol, 0.1 eq) and triethyl amine (8.4 g, 83.3 mmol, 7.0 eq) were added to a clean and dry reaction flask, and purged and protected with nitrogen. After cooling to −5° C. to 5° C., trifluoroacetyl chloride (10.2 g, 77.4 mmol, 6.5 eq) was slowly added dropwise. After that, the reaction was continued at room temperature for 8 hrs. The reaction endpoint was monitored by HPLC. After the reaction was completed, the reaction solution was diluted with dichloromethane (48 g, 15 ml), and washed once with a 2 N hydrochloric acid aqueous solution, once with a saturated sodium bicarbonate aqueous solution, and once with saturated brine. The organic phase was dried overnight. After filtration, the filtrate was transferred to a dry reaction flask, then diethylaminosulfur trifluoride (27 g, 168 mmol, 15.0 eq) and anhydrous ethanol (0.02 g, 4‰ M_{SM}) were added, heated to 20-25° C. and reacted for 72 hrs. The reaction solution was quenched with iced water, neutralized, and washed. The organic phase was separated, and concentrated to half of the volume. A 5% potassium carbonate aqueous solution (20 ml) was added and stirred at 20-25° C. for 3 hrs. After filtration, the filter cake was slurried in water and filtered again to obtain a crude product of Formula (VI). The crude product was added to acetonitrile (25 ml), and heated to reflux. After complete dissolution, the solution was slowly cooled, and crystallized for 2 hrs while the temperature was maintained at 40-45° C. The reaction solution was cooled to 0-10° C., and continuously stirred at 0-10° C. for 1 hr. After filtration, the filter cake was dried under vacuum at 50±3° C. for 8 hrs, to obtain a white solid as a solid (4.18 g, purity 98.0%, yield 79.3%).

### Example 3. Preparation of Compound V-1 where R is p-Methoxybenzyl

### 1) (2R,3S,4R)-2,3,4-tri(p-methoxybenzyloxy)-5-((p-methoxybenzyloxy)methyl)-4′-chloro-3′-(4-ethoxybenzyl)-1,2,3,4-tetrahydro-[1,1′-diphenyl]-1-ol (Formula I)

2-methyltetrahydrofuran (180 g, 202 ml) and then 4-iodo-1-chloro-2-(4-ethoxybenzyl) benzene (22.0 g, 59.04 mmol, 1.20 eq) were added to a clean and dry reaction flask, stirred until the solid was dissolved, purged with nitrogen, and cooled to −28 to −35° C. A 2.5 M solution of n-butyl lithium (17.28 g, 63.53 mmol, 1.30 eq) was added dropwise. After that, the reaction was continued at −25 to −30° C. further for 1 h. The remaining of the starting raw material was detected by HPLC. After the reaction was completed, (4R,5S,6R)-4,5,6-tris(p-methoxybenzyloxy)-3-((p-methoxybenzyloxy) methyl)cyclohex-2-en-1-one (32.0 g, 48.87 mmol, 1.0 eq) was dissolved in tetrahydrofuran (35 g), and added dropwise to the reaction flask at a temperature controlled to −28 to −35° C. After that, the reaction solution was stirred for 1 hr while the temperature was maintained, and the reaction progress was detected by HPLC. After the reaction was completed, the reaction solution was quenched by slowly adding to an ammonium chloride solution (220 g, 10%), and extracted by adding ethyl acetate (170 g). The organic phase was separated, washed with saturated sodium chloride (265 g), dried, and concentrated at 40-45° C. under reduced pressure until no fraction was flowed out. A brown oil (46.8 g, yield 106.2%) was obtained.

### 2) (2R,3S,4R,5R)-2,3,4-tri(p-methoxybenzyloxy)-5-((p-methoxybenzyloxy) methyl)-4′-chloro-3′-(4-ethoxybenzyl)-2,3,4,5-tetrahydro-1,1′-biphenyl (Formula II)

Dichloromethane (220 g, 170 ml) and then (2R,3S,4R)-2,3,4-tri(p-methoxybenzyloxy)-5-((p-methoxybenzyloxy)methyl)-4′-chloro-3′-(4-ethoxybenzyl)-1,2,3,4-tetrahydro-[1,1′-diphenyl]-1-ol (40 g, 44.37 mmol, 1.00 eq) were added to a clean and dry reaction flask, purged with nitrogen, and then cooled to −28 to −18° C. Triethylsilane (13.5 g, 116.1 mmol, 2.68 eq) was added dropwise. After that, boron trifluoride etherate (10.7 g, 75.38 mmol, 1.70 eq) was further added dropwise. Then, the reaction was continued for 1.5 hrs while the temperature was held. The reaction process was detected by HPLC. After the reaction was completed, the temperature was controlled to 10° C. or below. Purified water (80 g) was added to the reaction solution. The organic phase was washed to neutral with a saturated sodium bicarbonate aqueous solution (125 g), and then with saturated brine (125 g). The organic phase was collected. The organic phase was then concentrated at 37±2° C. under reduced pressure until no fraction was flowed out. The concentrated oil was added with methanol (152 g), heated to 70-75° C., then cooled to 10-13° C., and crystallized by stirring for 12 hrs. The product was filtered under suction, rinsed with methanol (45 g), and then further filtered under suction to obtain a wet product (28.3 g). The wet product was dried in the air at 50±3° C. for 15 hrs to obtain a pale yellow solid (23.57 g, purity 93.7%, yield 60.0%).

### 3) (1R,2S,3R,4R,5S,6R)-3,4,5-tri(p-methoxybenzyloxy)-2-((p-methoxybenzyloxy) methyl)-6-(4-chloro-3-(4-ethoxybenzyl)phenyl)cyclohexanol (Formula III)

Anhydrous tetrahydrofuran (180 g) and then (2R,3S,4R,5R)-2,3,4-tri(p-methoxybenzyloxy)-5-((p-methoxybenzyloxy) methyl)-4′-chloro-3′-(4-ethoxybenzyl)-2,3,4,5-tetrahydro-1,1′-biphenyl (23 g, 25.97 mmol, 1.00 eq) were added to a clean and dry reaction flask, and purged with nitrogen. After the system was completely dissolved, a solution of borane tetrahydrofuran complex (3.22 g, 40.26 mmol, 1.55 eq) was slowly added while the temperature was controlled to 20-24° C. After that, the reaction solution was slowly heated to 60-65° C. and reacted for 3 hrs. The reaction progress was detected by HPLC. After the reaction was completed, the reaction solution was cooled to 0-5° C., and a 5 mol/L solution of sodium hydroxide (55.6 g, 234.6 mmol, 9.03 eq) and then a 30% solution of hydrogen peroxide (26.1 g 230.3 mmol, 8.87 eq) were slowly added dropwise while the temperature was maintained. After that, the reaction solution was slowly heated to 25-30° C. and reacted further for 10 hrs. A saturated ammonium chloride solution (201 g) and a saturated sodium chloride solution (105 g) were added to the reaction solution and stirred for 1.5 hrs. The solution was allowed to stand still, and the organic phase was separated and collected. The aqueous phase was extracted once with ethyl acetate (102 g). The organic phases were combined. The organic phase was washed with a 10% Na₂SO₃ solution (108 g). The organic phase was collected, and had no color change when tested with potassium iodide test paper. The organic phase was concentrated at 40-45° C. under reduced pressure, until no fraction was flowed out. The resulting oil was added with isopropyl ether (45 g), stirred at 20-28° C. for 12 hrs, and filtered under suction. The wet product was collected and dried under vacuum at 45±2° C. for 16 hrs, to obtain an off-white solid (13.1 g, purity 92.8%, yield 55.8%).

### 4) Preparation of (2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone of Formula IV

Dichloromethane (75 g, 58 ml) and (1R,2S,3R,4R,5S,6R)-3,4,5-tri (p-methoxybenzyloxy)-2-((p-methoxybenzyloxy)methyl)-6-(4-chloro-3-(4-ethoxybenzyl) phenyl)cyclohexanol (10 g, 11.07 mmol, 1.00 eq) were added to a dry and clean reaction flask, and stirred until the solid was dissolved. The reaction solution was cooled to 5-12° C., added with 2,2,6,6-tetramethylpiperidine oxide (2.7 g, 17.28 mmol, 1.56 eq), and reacted for 5 hrs while the temperature was maintained at 10-15° C. The reaction progress was detected by HPLC. After the reaction was completed, the reaction solution was slowly added to an aqueous solution (78 mL) containing anhydrous Na₂SO₃ (7.0 g) and NaHCO₃ (5.3 g), and stirred for 30 min. The solution was allowed to stand still, and the organic phase was separated and collected. The aqueous phase was extracted with dichloromethane (42 g). The organic phases were combined. The organic phase was washed with purified water (38 g). The organic phase was separated and, collected, dried over anhydrous sodium sulfate (5.9 g) for 30 min, and filtered. The filtrate was collected, and concentrated under reduced pressure at 30±5° C., to obtain a yellowish-brown oil. Isopropyl alcohol (16.5 g) was added to the oil, heated to 68-75° C. to dissolve it, then slowly cooled, and crystallized by stirring for 8 hrs while the temperature was maintained at 20-25° C. The reaction solution was filtered. The material was collected and dried in a vacuum oven at 50±3° C. for 10 hrs, to obtain (2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone (7.5 g, purity 94.8%, yield 75.2%).

### 5) Preparation of (2S,3S,4R,5R,6R)-2-(4-chloro-3-(4-ethoxybenzyl) phenyl)-3, 4, 5-trihydroxy-6-(hydroxymethyl) cyclohexan-1-one of Formula V-1

(2R,3R,4R,5S,6S)-3,4,5-tri(p-methoxybenzyloxy)-2-((p-methoxybenzyloxy)methyl)-6-(4-chloro-3-(4-ethoxybenzyl)phenyl)cyclohexanone (5 g, 5.55 mmol, 1.00 eq), tetrahydrofuran (20 g, 22 ml), and methanol (15 g, 20 ml) were added to a 100 mL hydrogenation reactor, and stirred until the solid was completely dissolved. Then, 10% palladium hydroxide on carbon (0.5 g) was added and stirred until uniform. Stirring was started at a speed of 25 Hz. The reactor was purged three times with nitrogen and then three times with hydrogen. The temperature was controlled at 20-25° C., the hydrogen pressure was 0.09-0.12 MPa, and the reaction time was 60 min. The reaction progress was detected by HPLC. After the reaction was completed, hydrogen was vented, and the reactor was purged three times with nitrogen. Palladium on carbon was removed by filtration, and the filter cake was washed with tetrahydrofuran (5.5 g). The filtrates were combined, concentrated to dryness under reduced pressure at 45±2° C., and acetonitrile (12 g) were added and heated to reflux. After complete dissolution, the solution was slowly cooled, and crystallized for 1 hr while the temperature was maintained at 40-44° C. The crystallization was continued by cooling to 0-5° C., with stirring at 0-5° C. for 1 hr. After filtration, the filter cake was dried under vacuum at 50±2° C. for 8 hrs, to obtain a white solid as a solid (1.85 g, purity 99.3%, yield 79.2%).

### 6) Preparation of (1R,2R,3S,4S,6R)-4-(4-chloro-3-(ethoxybenzyl) phenyl)-5,5-difluoro-6-(hydroxymethyl)cyclohexan-1,2,3-triol of Formula VI

(2S,3S,4R,5R,6R)-2-(4-chloro-3-(4-ethoxybenzyl) phenyl)-3, 4, 5-trihydroxy-6-(hydroxymethyl) cyclohexan-1-one (5 g, 11.9 mmol, 1.0 eq), dichloromethane (48 g, 15 ml), DMAP (0.18 g, 2.4 mmol, 0.2 eq) and triethyl amine (8 g, 65.5 mmol, 5.5 eq) were added to a clean and dry reaction flask, and purged and protected with nitrogen. After cooling to 0° C. to 5° C., trifluoroacetyl chloride (7.9 g, 59.5 mmol, 5.0 eq) was slowly added dropwise. After that, the reaction was continued at room temperature for 10 hrs. The reaction endpoint was monitored by HPLC. After the reaction was completed, the reaction solution was diluted with dichloromethane (32 g, 10 ml), and washed once with a 2 N hydrochloric acid aqueous solution, once with a saturated sodium bicarbonate aqueous solution, and once with saturated brine. The organic phase was dried overnight. After filtration, the filtrate was transferred to a dry reaction flask, then diethylaminosulfur trifluoride (27 g, 168 mmol, 15.0 eq) and anhydrous ethanol (0.01 g, 2‰ M_{SM}) were added, heated to 20° C. and reacted for 72 hrs. The reaction solution was quenched with iced water, neutralized, and washed. The organic phase was separated, and concentrated to half of the volume. A 5% potassium carbonate aqueous solution (10 ml) was added and stirred at 25-30° C. for 3 hrs. After filtration, the filter cake was slurried in water and filtered again to obtain a crude product of Formula (VI). The crude product was added to acetonitrile (20 ml), and heated to reflux. After complete dissolution, the solution was slowly cooled, and crystallized for 2 hrs while the temperature was maintained at 40-50° C. The reaction solution was cooled to 0-10° C., and continuously stirred at 0-10° C. for 1 hr. After filtration, the filter cake was dried under vacuum at 50±3° C. for 8 hrs, to obtain a white solid as a solid (4.13 g, purity 99.0%, yield 78.3%).

Various modifications and changes can be made to the compounds and methods of the present invention by ordinary technicians without deviating from the principle and spirit of the present invention to achieve the same technical effect, which are all contemplated in the same or equivalent scope as defined by the claims of the present invention.

1-11. (canceled)

12. An SGLT2 inhibitor intermediate V-1, having a structural formula below:

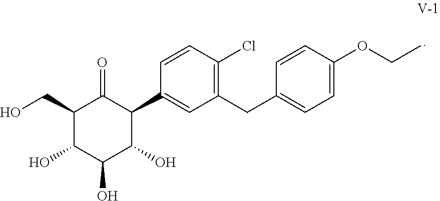 [SMILES: CCOc1ccc(Cc2cc([C@@H]3C(=O)[C@H](CO)[C@@H](O)[C@H](O)[C@H]3O)ccc2Cl)cc1]

13. A method for preparing an SGLT2 inhibitor intermediate V-1 according to claim 12, 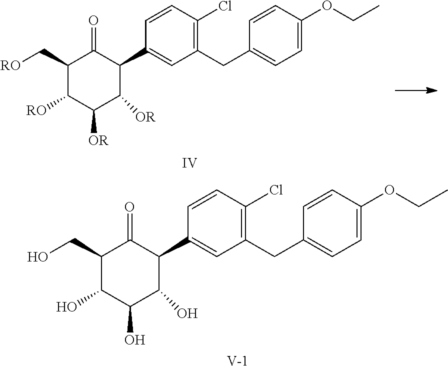
wherein R is selected from p-methoxybenzyl, benzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl;
the method comprising adding a compound of Formula IV and a deprotecting reagent into a solvent, reacting at 10-50° C. for 1-10 hrs, and then carrying out post-treatment, solvent removal, and refining after the reaction is completed to prepare the compound of Formula V-1, that is, (2S,3S,4R,5R,6R)-2-(4-chloro-3-(4-ethoxybenzyl) phenyl)-3, 4, 5-trihydroxy-6-(hydroxymethyl) cyclohexan-1-one.

14. The method according to claim 13, wherein the deprotecting reagent is one of palladium on carbon, palladium hydroxide on/carbon, tetrabutylammonium bromide, 2,3-dichloro-5,6-dicyanobenzoquinone, hydrogen chloride in methanol, and trifluoroacetic acid;
wherein the amount of the deprotecting reagent is 5-100 wt % of the amount of the compound of Formula IV;
wherein the solvent is one of tetrahydrofuran, methanol, dichloromethane, ethanol or acetone or any combination thereof;
wherein the ratio of (2R,3R,4R,5S,6S)-3,4,5-tris(R protecting group-oxy)-2-((R protecting group-oxy)methyl)-6-(4-chloro-3-(4-ethoxybenzyl)phenyl)cyclohexanone to the solvent is 1 g/(5-12) ml; and
wherein the reagent used for refining is methanol, isopropanol, ethanol, acetone, ethyl acetate or acetonitrile.

15. A method comprising using the SGLT2 inhibitor intermediate V-1 according to claim 13 in the preparation of an SGLT2 inhibitor of Formula (VI):

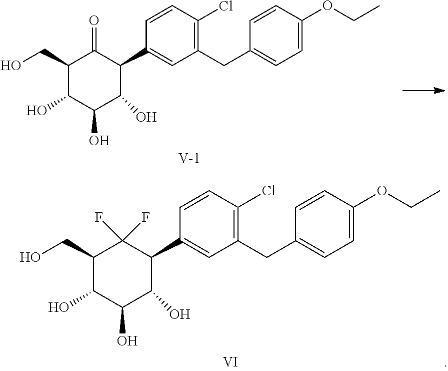

16. The method according to claim 15, wherein the method comprises: adding the intermediate V-1, a catalyst and triethylamine into a solvent, adding trifluoroacetyl chloride at −5 to 5° C., reacting at room temperature, washing, drying, filtering, then adding diethylaminosulfur trifluoride and anhydrous ethanol, reacting at 15-25° C. for 60-80 hrs, adding a potassium carbonate aqueous solution, and reacting at 20-30° C. for 3-5 hrs, to obtain the compound VI.

17. The method according to claim 16, wherein the catalyst in the synthesis of the compound of Formula VI is selected from DMF or DMAP;
wherein the molar ratio of the intermediate V-1, the catalyst and triethylamine in the synthesis of the compound of Formula VI is 1:(0.1-0.5):(6-8);
wherein the molar ratio of the intermediate V-1 to trifluoroacetyl chloride in the synthesis of the compound of Formula VI is 1:(5-7);
wherein the molar ratio of the intermediate V-1 to diethylaminosulfur trifluoride in the synthesis of the compound of Formula VI is 1:(10-20); and
wherein the anhydrous ethanol is added in an amount of 1‰-5‰ by weight of the amount of the intermediate V-1 in the synthesis of the compound of Formula VI.

18. A method for preparing an SGLT2 inhibitor of Formula VI: 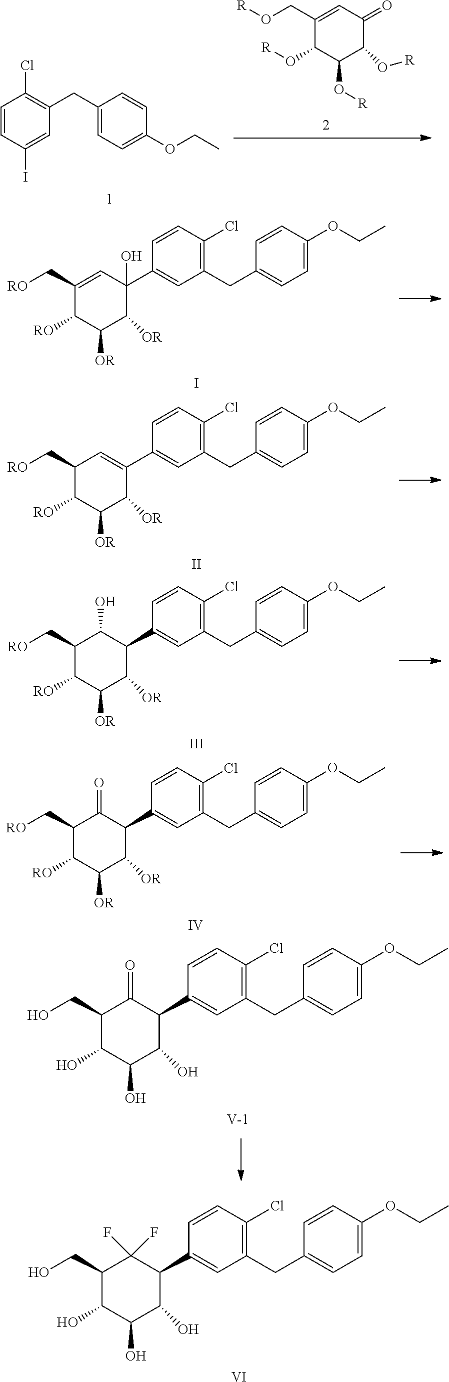
wherein R is selected from methoxybenzyl, benzyl, triphenylmethyl, acetyl, benzoyl, pivaloyl, trimethylsilyl, t-butyldimethylsilyl, t-butyldiphenylsilyl, tri-iso-propylsilyl, 2-tetrahydropyranyl, methoxymethyl or 2-ethoxyethyl.

19. The method according to claim 18, wherein a method for synthesizing the compound of Formula I comprises specifically: adding 4-iodo-1-chloro-2-(4-ethoxybenzyl)benzene (material 1), a material 2, and a metal reagent to a solvent at a molar ratio of (1.05-1.25):1.0:(1.0-1.5), and reacting at −20 to −70° C. for 1-3 hrs.

20. The method according to claim 19, wherein the molar ratio of 4-iodo-1-chloro-2-(4-ethoxybenzyl)benzene (material 1), the material 2, and the metal reagent in the synthesis of the compound of Formula I is (1.1-1.2):1:(1.3-1.35);
wherein the reaction temperature in the synthesis of the compound of Formula I is −25 to −35° C.;
wherein the metal reagent in the synthesis of the compound of Formula I is isopropyl magnesium chloride-lithium chloride or n-butyl lithium; and
wherein the solvent used in the synthesis of the compound of Formula I is one of methyl t-butyl ether, tetrahydrofuran, 2-methyltetrahydrofuran and toluene, or a combination thereof; preferably, the solvent is tetrahydrofuran or 2-methyltetrahydrofuran; and further preferably, the ratio of the material 2 to the solvent is 1 g/(5-10) ml.

21. The method according to claim 18, wherein a method for synthesizing the compound of Formula II comprises specifically: adding the compound of Formula I, triethylsilane, and boron trifluoride etherate to a solvent at a molar ratio of 1.0:(1.5-3.5):(1.1-2.0), reacting at −30 to 0° C. for 1-2 hrs, washing the reaction solution with alkaline water after the reaction is completed, discarding the aqueous layer, removing the solvent, and refining to prepare the compound of Formula II.

22. The method according to claim 21, wherein the molar ratio of the compound of Formula I, triethylsilane, and boron trifluoride etherate in the synthesis of the compound of Formula II is 1.0:(2.1-2.7):(1.45-1.8);
wherein the reaction temperature in the synthesis of the compound of Formula II is −28 to −15° C.;
wherein the solvent used in the synthesis of the compound of Formula II is isopropyl acetate, cyclohexane, dichloromethane, isopropyl ether or acetonitrile, and preferably the solvent is dichloromethane; and in some embodiments, the ratio of the compound of Formula I to the solvent is 1 g/(2-15) ml;
wherein the alkaline water in the synthesis of the compound of Formula II is a sodium bicarbonate solution, a sodium carbonate solution or a sodium hydroxide solution, and preferably a sodium bicarbonate solution; and
wherein the reagent used for refining in the synthesis of the compound of Formula II is one of acetonitrile, isopropanol, methanol, ethanol, isopropyl ether, ethyl acetate or n-heptane, or a combination thereof.

23. The method according to claim 18, wherein a method for synthesizing the compound of Formula III specifically comprises: adding the compound of Formula II and a reducing agent to a solvent at a molar ratio of 1.0:(1.1-2.5), and reacting at 40-70° C. for 1-7 hrs; cooling the reaction solution to −5 to 30° C. after the reaction is completed, and further oxidizing for 12-15 hrs at 15-40° C. at a molar ratio of the compound of Formula II:sodium hydroxide:H₂O₂ of 1.0:(3-10):(3-10), followed by washing, extraction, concentration, and refining of the reaction solution, to obtain the compound of Formula III.

24. The method according to claim 23, wherein in the synthesis of the compound of Formula III, the molar ratio of the compound of Formula II to the reducing agent is 1.0:(1.4-1.6);
wherein the reaction temperature in the synthesis of the compound of Formula III is 60-70° C.;
wherein the molar ratio of the compound of Formula II:sodium hydroxide:H₂O₂ in the synthesis of the compound of Formula III is 1.0:(7.5-9.5):(8.0-9.0);
wherein the further oxidation reaction temperature in the synthesis of the compound of Formula III is 25 to 33° C.;
wherein the reducing agent in the synthesis of the compound of Formula III is borane dimethyl sulfide, sodium borohydride, pinacol borane, 8-methoxy-9-borabicyclic[3.3.1] nonane or borane tetrahydrofuran complex, and preferably borane dimethyl sulfide or borane tetrahydrofuran complex;
wherein the solvent used in the synthesis of the compound of Formula III is one of toluene, tetrahydrofuran, 1,4-dioxane and dichloromethane, or a combination thereof, and preferably tetrahydrofuran; and preferably, the ratio of the compound of Formula II to the solvent is 1 g/(6-14) ml; and
wherein the reagent used for refining in the synthesis of the compound of Formula III is one of acetonitrile, n-heptane, methyl tert-butyl ether, ethanol, isopropyl ether and ethyl acetate or a combination thereof.

25. The method according to claim 18, wherein a method for synthesizing the compound of Formula IV comprises specifically: adding the compound of Formula III and an oxidant to a solvent at a molar ratio of 1.0:(1.0-4.0), and reacting at −5 to 30° C. for 1-5 hrs; and washing the reaction solution with water containing a reducing agent after the reaction is completed, discarding the aqueous layer, removing the solvent, and refining to produce the compound of Formula IV.

26. The method according to claim 25, wherein in the synthesis of the compound of Formula IV, the molar ratio of the compound of Formula III to the oxidant is 1.0:(1.5-1.8);
wherein the reaction temperature in the synthesis of the compound of Formula IV is 5 to 15° C.;
wherein the oxidant used in the synthesis of the compound of Formula IV is manganese dioxide, Dess-Martin oxidant, Jones reagent or 2,2,6,6-tetramethylpiperidine oxide;
wherein the solvent used in the synthesis of the compound of Formula IV is one of isopropyl ether, dichloromethane, tetrahydrofuran, toluene, and acetonitrile, or a combination thereof, and preferably dichloromethane; and in some embodiments, the ratio of the compound of Formula III to the solvent is 1 g/(3-15) ml; and
wherein the reagent used for refining in the synthesis of the compound of Formula IV is isopropyl ether, isopropyl alcohol, ethanol, acetone, ethyl acetate or tetrahydrofuran.

27. The method according to claim 18, wherein a method for synthesizing the compound of Formula V-1 comprises specifically: adding the compound of Formula IV and a deprotecting reagent to a solvent, reacting at 10-50° C. for 1-10 hrs, and then carrying out post-treatment, solvent removal, and refining after the reaction is completed to obtain the compound of Formula V-1.

28. The method according to claim 27, wherein the deprotecting reagent in the synthesis of the compound of Formula V-1 is one of palladium on carbon, palladium hydroxide on carbon, tetrabutylammonium bromide, 2,3-dichloro-5,6-dicyanobenzoquinone, hydrogen chloride in methanol, and trifluoroacetic acid;
wherein the amount of the deprotecting reagent used in the synthesis of the compound of Formula V-1 is 5-100 wt % of the amount of the compound of Formula IV;
wherein the solvent in the synthesis of the compound of Formula V-1 is one of tetrahydrofuran, methanol, dichloromethane, ethanol or acetone or any combination thereof;
wherein in the synthesis of the compound of Formula V-1, the ratio of the compound of Formula IV to the solvent is 1 g/(5-12) ml; and
wherein the reagent used for refining in the synthesis of the compound of Formula V-1 is methanol, isopropyl alcohol, ethanol, acetone, ethyl acetate or acetonitrile.

29. The method according to claim 18, wherein a method for synthesizing the compound of Formula VI specifically comprises adding the intermediate V-1, a catalyst and triethylamine into a solvent, adding trifluoroacetyl chloride at −5 to 5° C., reacting at room temperature, washing, drying, filtering, then adding diethylaminosulfur trifluoride and anhydrous ethanol, reacting at 15-25° C. for 60-80 hrs, adding a potassium carbonate aqueous solution, and reacting at 20-30° C. for 3-5 hrs, to obtain the compound VI.

30. The method according to claim 27, wherein the catalyst in the synthesis of the compound of Formula VI is selected from DMF or DMAP;
wherein the molar ratio of the intermediate V-1, the catalyst and triethylamine in the synthesis of the compound of Formula VI is 1:(0.1-0.5):(6-8), and preferably 1:(0.1-0.2):(6-8);
wherein the molar ratio of the intermediate V-1 to trifluoroacetyl chloride in the synthesis of the compound of Formula VI is 1:(5-7);
wherein the molar ratio of the intermediate V-1 to diethylaminosulfur trifluoride in the synthesis of the compound of Formula VI is 1:(10-20), and preferably 1:15;
and wherein anhydrous ethanol is added in an amount of 1‰-5‰ by weight of the amount of the intermediate V-1 in the synthesis of the compound of Formula VI.
